(19) United States
(12) Patent Application Publication
(10) Pub. No.: US 2024/0252104 A1
(43) Pub. Date: Aug. 1, 2024

(54) SYSTEM AND METHOD FOR EARLY DIAGNOSIS OF SKELETON SYNDROMES

(71) Applicant: Kedi Mobil Uygulama Anonim Sirketi, MUGLA (TR)

(72) Inventors: Banu KILIC, Istanbul (TR); Tugba SUZEK, Mugla (TR)

(73) Assignee: Kedi Mobil Uygulama Anonim Sirketi, MUGLA (TR)

(21) Appl. No.: 18/681,080

(22) PCT Filed: Nov. 22, 2021
(86) PCT No.: PCT/TR2021/051268

§ 371 Date: Feb. 5, 2024

(30) Foreign Application Priority Data
Nov. 23, 2020 (TR) 2020/18758
Nov. 21, 2021 (TR) 2021/018150

(51) Int. Cl. A61B 5/00; A61B 5/107; G16H 50/20

(52) U.S. Cl. CPC A61B 5/4542; A61B 5/0077; A61B 5/1071; A61B 5/1072; A61B 5/1077; A61B 5/7267; G16H 50/20; A61B 2562/0219

(57) ABSTRACT

A system comprising a user terminal for diagnosis of skeleton syndromes in which the skeleton characteristics of a person to be diagnosed are visible from the profile is provided. The user terminal comprises a camera to capture a diagnosis image seen from the side of the face of the person to be diagnosed and a processing unit associated with the camera. The processing unit is configured to determine a mandible area and a maxillary area in the diagnosis image received from the camera, calculate an area ratio that is the ratio of one of the surface area ratios of the maxillary area and the surface area of the jaw area to the other and produce a signal related to the skeleton syndromes of the person to be diagnosed in accordance with a deviation of the calculated area ratio from a predetermined threshold value.

### CROSS REFERENCE TO THE RELATED APPLICATIONS

This application is the national phase entry of International Application No. PCT/TR2021/051268, filed on Nov. 22, 2021, which is based upon and claims priority to Turkish Patent Application No. 2020/18758, filed on Nov. 23, 2020, and Turkish Patent Application No. 2021/018150, filed on Nov. 21, 2021, the entire contents of which are incorporated herein by reference.

### TECHNICAL FIELD

The invention relates to a system and method computer-based for diagnosis of skeleton syndromes in which the skeleton characteristics are visible from the profile, such as Skeleton Class III malocclusion disease, Hutchinson Gilford Progeria syndrome, Wegener's granulomatosis, Nager Syndrome, Down syndrome.

### BACKGROUND

Skeleton Class III malocclusion disease, Hutchinson Gilford Progeria syndrome, Wegener's granulomatosis, Nager Syndrome, Down syndrome are the skeleton syndromes in which the skeleton characteristics are visible from the profile.

The early diagnosis of the treatable ones among the above-mentioned skeleton syndromes is important.

Skeleton Class III malocclusion is a condition characterized by the sagittal developmental retardation and/or rearward positioning of the maxilla or the extreme development and/or forward positioning of the mandible. The correction of the harmony between the mandible and maxilla can be provided by orthodontic/orthopedic operations to be performed in early terms on the patients affecting the active sutures unclosed. The problem can be masked with the orthodontic treatment if it is detected in early ages, while the surgery may be the only option in advanced ages. The correction of skeleton Class III malocclusion in a growing patient by an orthopedic device in early terms is very important since it can eliminate a future surgical need, in addition since the operation can only be performed in adulthood, the early treatment also helps with preventing the negative effects of a facial deformity on the social life of a childhood patient.

In the present art, the early diagnosis of skeleton syndromes is performed by examination. The main reason for this problem is the high orthodontic examination fees, the insufficient number of available orthodontists with respect to the total population, and the fact that children are starting to see a dentist after permanent dentition is a false common belief in society. Therefore in the present art, there is a need for cost-effective and easy access methods for the diagnosis of skeleton Class III malocclusion.

In the patent document numbered ES2166675, in the head film image of a patient, markers are placed at the points on the patient's face and the angles of these points with respect to a true vertical line are calculated. The true vertical line, as it is known in the art, is a line passing through predetermined points in a side profile view of the person's head and running vertically.

Consequently, all of the above-mentioned problems have made it necessary to make an innovation in the related technical field.

### SUMMARY

The present invention relates to a system for diagnosis of skeleton syndromes for the purpose of eliminating the above-mentioned disadvantages and providing new advantages to the related art.

An aim of the invention is to propose a system enabling to diagnose and estimate skeleton syndromes without going to a clinic and without the need for an expert as well as aiding to diagnose the identities of the persons.

Another aim of the invention is to propose a system enabling to diagnose skeleton class III malocclusions with increased accuracy.

For purpose of achieving all of the aims above mentioned and the ones to have appeared from the detailed description below, the present invention is a system comprising a user terminal for diagnosis of skeleton syndromes in which the skeleton characteristics of a person to be diagnosed are visible from the profile. Accordingly, said user terminal comprises a camera to capture a diagnosis image seen from the side of the face of the person to be diagnosed and a processing unit associated with the said camera; said processing unit is configured to determine a mandible area and a maxillary area in the diagnosis image received from the camera, calculate an area ratio that is the ratio of one of the surface area ratio of said maxillary area and the surface area of said jaw area to the other, produce a signal related to the skeleton syndromes of the person to be diagnosed in accordance with a deviation of the calculated area ratio from a predetermined threshold value. Thus the skeleton class III malocclusions can be diagnosed without the need for an expert.

A possible embodiment of the invention is characterized in that said processing unit is configured to enable to view the information related to the identity of the matched person on a user interface if the determined area ratio matches with one of the area ratios that belong to recorded persons in the memory unit. The jaw structures of the persons are characteristics and it may take some time to change that. By means of this system, law enforcement officers can identify people whose chin characteristic matches the chin characteristic of a wanted person.

The invention is also a system comprising a user terminal for diagnosis of skeleton syndromes in which the skeleton characteristics of a person to be diagnosed are visible from the profile. Accordingly, the novelty of the invention is that said user terminal comprises a camera to capture a diagnosis image seen from side of the face of the person to be diagnosed, a processing unit associated with said camera, a user interface controlled by said processing unit and an acceleration sensor; said processing unit is configured to determine a mandible area in the diagnosis image received from the camera; calculate an area ratio that is the ratio of one of the surface area ratio of said maxillary area and the surface area of said jaw area to the other; determine a true vertical line running in parallel to gravitational force in accordance with the measurements received from the acceleration sensor and enable to view it on said user interface; determine in the diagnosis image received from the camera a plurality of points, at least one on the mandible and at least one on the maxilla; determine the point distances of the determined points to said true vertical line; determine an H angle between a first line passing through two of the determined points and a second point also passing through two of the determined points and having a common point with the first point;

the processing unit is also configured to produce a signal indicating, by means of a machine learning model trained with side profile images that belong to a plurality of healthy persons and a plurality of persons suffering from skeleton syndromes of which the H angles, the point distances and the area ratios are known, whether the diagnosis image contains skeleton syndromes in accordance with the determined H angle, the point distances and the area ratio in the diagnosis image.

The invention is also a system comprising a user terminal for diagnosis of skeleton syndromes in which the skeleton characteristics of a person to be diagnosed are visible from the profile. Accordingly, the novelty of the invention is that said user terminal comprises a camera to capture a diagnosis image seen from the side of the face of the person to be diagnosed and a processing unit associated with the said camera; said processing unit is configured to transform the received image to a silhouette image, divide the silhouette image into a predetermined number of sub-images and transform the obtained sub-images to a binary vector or matrix, produce a signal related to the skeleton syndromes of the person to be diagnosed by using a deep machine learning model trained with the side profile images in the form of transformed images of persons suffered from skeleton syndromes and healthy persons.

### BRIEF DESCRIPTION OF THE DRAWINGS

FIG. 1 shows a representative view of the system.

FIG. 2 shows a representative view of the system.

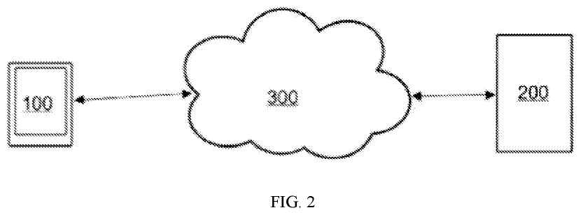

FIG. 3 shows a representative view of the user terminal.

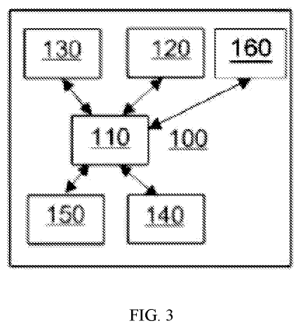

FIG. 4 shows an image of the person to be diagnosed taken with a camera and a representative view of the detected points and areas.

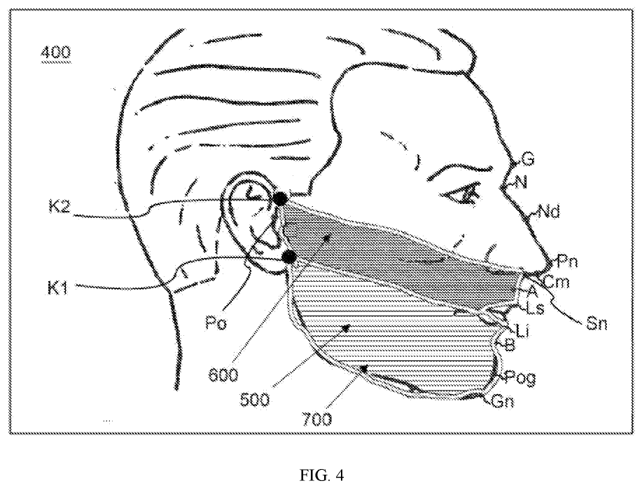

FIG. 5 shows an image of the person to be diagnosed taken with a camera and a representative view of the detected points and the distances thereof to a true vertical line.

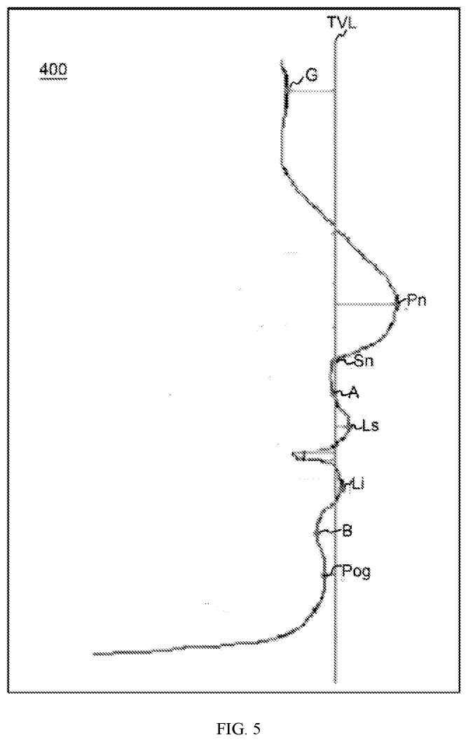

FIG. 6 shows the diagnosis image in a silhouette form.

### DETAILED DESCRIPTION OF THE EMBODIMENTS

In this detailed description, the subject of the invention is described with non-limiting examples for merely a better understanding of the subject.

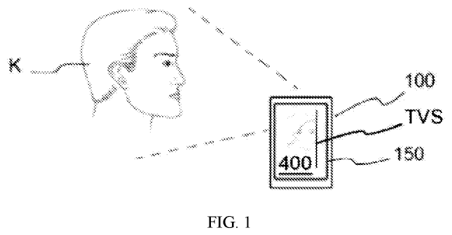

By referring to FIG. 1, the invention is a system comprising a user terminal to capture an image of the person for identifying persons and diagnosis of skeleton syndromes in which the skeleton characteristics of a person to be diagnosed are visible from the profile. This system essentially takes a side profile image of the person, detect predetermined points in the profile images of the persons, determine a jaw area and a maxillary area defined by some of these points, classify the skeleton syndromes of the person in accordance with the deviation of the surface area of jaw area and maxillary area from the predetermined threshold values. In addition, it can also help with identifying a person.

As another inventive aspect of the invention, the distances of the points from a true vertical line are determined, the skeleton syndrome of the person in the diagnosis image by means of a machine learning model trained by using as characteristics, the angle made by a first line and a second line passing through some of these points and the above-mentioned areas.

The training data mentioned herein comprises side view images of the persons. The side view images of the persons can comprise the above-mentioned points. More specifically, the images in training data can be associated with the persons' identities and their skeleton syndromes. The images can comprise the determined points, the distances of these points to said true vertical line, their area ratios and as well as the ratio between the distances to the true vertical line.

As a result of the above-mentioned classification, it can be provided that the person whose image has been taken matches one of the persons in the training data or match a skeleton syndrome of one of the persons in the training data. Therefore by means of the invention, a helpful tool is provided for law enforcement officers in order to find the individual best matched to the individual from the forensic records, and also the diagnosis of skeleton syndromes of persons can be provided.

More specifically, the system comprises a user terminal (100). The user terminal (100) comprises a camera (130) for the user to take a photo from the side of the person whose syndrome (for example, skeleton class III malocclusion) to be diagnosed, as well as a processing unit (110) controlling said camera (130) and receiving a diagnosis image (400) captured by the camera (130) as an input. Said processing unit (110) is associated with a memory unit (120) so that it can read and write data. The memory unit (120) further comprises both software consisting of command lines executed by the processing unit (110) and the machine learning models. When executed by the processing unit (110) said software ensures the process steps enabling the system of the invention to identify to be performed. Said user terminal (100) can comprise a user interface (150) and enable the user to input commands to the processor and the processor to present data to the user. The user terminal (100) can further connect to a communication web (300) by means of a communication unit (140) and can exchange data with a server (200). The machine learning models (300) can also be present in the server. The user terminal (100) can be a mobile phone, a tablet computer, a general-purpose computer, etc. The processing unit (110) performing the characterizing steps of the system subject of the invention can be provided in the server (200) or user terminal (100). An image received from the user terminal (100) can be sent to the server (200) and processed in the server (200).

In an embodiment of the invention, the user terminal (100) comprises an acceleration sensor (160). The processing unit (110), in accordance with the signal it receives from the acceleration sensor (160), enables a true vertical line parallel to the gravitational force, i.e. orthogonal to the floor to be viewed on the user interface (150) during the image capturing. The true vertical line (TVL) is also known as the “true vertical line” in the art (orthodontics). The processing unit (110), in accordance with the determined points and determined true vertical line, can enable the instructions to be viewed on the user interface in order to enable the user to position the true vertical line (TVL) correspondingly.

The processing unit (110) receives a diagnosis image (400) in which the user's face is seen in a side view from the camera (130) as an input. It detects the predetermined points on the diagnosis image (400) which it receives as an input. The processing unit (110) can use image rendering and face recognition algorithms known in the art during this process.

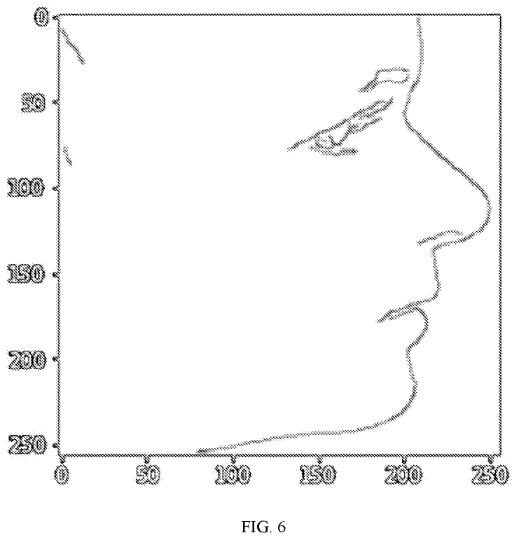

By referring to FIG. 6, the points mentioned here are defined as a G point (G) remained in between the brows of the person to be diagnosed (K); a Pn point (Pn) near the peak point of the nose of the person to be diagnosed (K); an Sn point (Sn) where the wall between the nostrils of the person to be diagnosed (K) meet the maxilla; an Ls point (Ls) that is an initial point on the side of the upper lip facing to nose of the person to be diagnosed (K); an Li point (Li) on the initial point of the lower lip facing to jaw; a Pog point (Pog) near the peak point of the jaw and the Sn point (Sn) at base of the cavity between the Pog point (Pog) and the Li point (Li); an N point (N) at base of the cavity between the forehead and the nose, an Nd point (Nd) that is the middle of the nasal bridge; a Cm point (Cm) between the Sn point (Sn) and the Pn point (Pn); an A point (A) between the Ls point (Ls) and the Sn point (Sn); a B point (B) at base of the cavity between the Li point (Li) and the Pog point (Pog); a Gn point (Gn) at the lower edge of the jaw and a Po point (Po) at the ear entry. Each of these points is well known by the names they are given in orthodontics. Therefore, more detailed information about the locations of these points will not be given in this detailed description. In addition, in this description, the lower point where the ear meets the face is defined as a lower ear point (K1) and the upper point where the ear meets with the face is defined as an upper ear point (K2).

The edge limits thereof are defined as a mandible area comprising the mandible bone, the Gn point (Gn), the Pog point (Pog), the B point (B), the Li point (Li), the lower ear point (K1) and the upper ear point (K2). It contains a near-line edge between the upper ear point (K2) and the Li point (Li). The edge limits thereof are defined as a maxillary area having a rectangular like are comprising the Li point (Li), the point where two lips meet, the Ls point (Ls), the A point (A), the Sn point (Sn), the lower ear point (K1) and the upper ear point (K2). The maxillary area and the mandible area are well known in art. The processing unit (110) determines the jaw area (500) and maxillary area (600) in the diagnosis image (400).

Accordingly, the processing unit (110) determines the above-mentioned point in the diagnosis image (400) it receives. For determining these points, the processing unit can use image rendering techniques known in the art. Accordingly, it can detect the nose, chin, lips, forehead, etc. and can determine the points properly according to their location in the image. The object recognition and face recognition techniques will not be described in detail since they are well known in art.

Then, the processing unit determines a jaw area (500) and a maxillary area (600) according to the points it detects. The processing unit (110) determines an area ratio that is the surface area ratio of one of the jaw area (500) and maxillary area (600) to the other. The processing unit produces a signal related to skeleton syndrome if it detects the determined area ratio deviates from a predetermined threshold value. In a possible embodiment of the invention, it questions whether there is a match with one of the side profile images in the training, and if there is a match, produces a signal related to the identity information of this person. Therefore, law enforcement officers can use the invention for identifying persons.

Studies have shown that people of different ethnicities differ in the detection points and area ratios. The above-mentioned training data can be divided into different ethnic groups, in case the ethnic group of the person whose image is taken is received as an input, at first, it can be classified by a machine learning model trained with data containing this ethnic group.

In this embodiment, the mentioned skeleton syndromes may be Skeleton Class III malocclusion disease, Hutchinson Gilford Progeria syndrome, Wegener's granulomatosis, Nager Syndrome, Down syndrome, etc. The examples given in this detailed description are related to Skeleton Class III malocclusion disease.

In another possible embodiment of the invention, the processing unit (110) detects a plurality of point distances between the detected points, a plurality of points and said true vertical line (TVL). The points mentioned herein may be G point (G), Sn point (Sn), A point (A), Ls point (Ls), Li point (Li), B point (B) and Pog point (Pog). Then, the processing unit (110) detects a first line passing through the Sn point (Sn) and the Pog point (Pog) a second line passing through the N point (N) and the Pog point (Pog). Then, the processing unit (110) detects an angle H between the first line and the second line. The processing unit (110) enables the diagnosis image to be classified by means of a machine learning model trained with the side profile views whose area ratio, H angle and point distance are known. According to the classifying result, one of the following results can be obtained; the person in the diagnosis image matches a person in the data used in the training, the person is healthy or suffers from a skeleton syndrome. This information can be viewed on the user interface.

The machine learning model used herein may be the random forest model. The H angle, area ratio, point distances, the ratio of the point distances to each other and to TVL line can be used as characteristics.

Then, the processing unit (110) performs the classification via machine learning by using this ratio. The model mentioned herein is a machine learning model trained with a plurality of side profile photos in which the persons suffering from skeleton syndrome and healthy persons, whose maxillary area (600) and jaw areas (500) are determined and/or area ratios are determined are involved. The processing unit (110) acquires via the machine learning model the classification result indicating whether the diagnosis image belongs to a healthy individual or individual suffering from a skeleton syndrome.

In a possible embodiment of the invention, the images of the persons who are healthy or have been diagnosed with a skeleton syndrome as a result of the diagnosis and their information can be fed to the machine learning model as training data; thereby the system can continuously update itself and improve the diagnosis it makes.

In another possible embodiment of the invention, the processing unit transforms the diagnosis image to a silhouette form thereof. The silhouette form mentioned herein is an image that contains the pixels showing only the outer edges of the head of the person in the image. It is well known in art. Then, the processing unit divides the image in form of a silhouette into 255 parts, transforms it into a matrix, and then transforms this matrix into a binary vector.

A deep learning model is trained with transforming the images of the persons suffering from skeleton syndromes and healthy persons into a silhouette form and with training data comprising the binary vector obtained therefrom. The deep learning model is trained with dividing the data as a train test, transforming the train data set into a one-dimensional sequence by using the keras library with Keras Flatten, creating 4 layers with Keras by using rectified linear units (relu) in input layers, Sigmoid in Hiden layers and Sigmoid activation function in Output layer, using Adam optimizer as an optimizer and using sparse_categorical_crossentropy as accuracy and lost function for the matrix. The skeleton syndrome classification is performed by using this model.

The protection scope of the invention has been determined in the annexed claims and should not be limited with the fact explained by way of example in this detailed description. Of course, it is obvious that a person skilled in art can make similar embodiments in the light of the above-explained facts without departing from the principle of the invention.

What is claimed is:

1. A system for diagnosis of skeleton syndromes, wherein skeleton characteristics of a person to be diagnosed are visible from a profile; comprising a user terminal, wherein the user terminal comprises a camera to capture a diagnosis image seen from the side of a face of a person to be diagnosed and a processing unit associated with the camera; the processing unit is configured to determine a mandible area and a maxillary area in the diagnosis image received from the camera, calculate an area ratio that is a ratio of one of the surface area ratios of the maxillary area and the surface area of the jaw area to the other, produce a signal related to skeleton syndromes of the person to be diagnosed in accordance with a deviation of the calculated area ratio from a predetermined threshold value.

2. The system for diagnosis of skeleton syndromes according to the claim 1, wherein the processing unit is configured to determine a G point remained in between the brows of the person to be diagnosed; a Pn point near a peak point of the nose of the person to be diagnosed; an Sn point where the wall between the nostrils of the person to be diagnosed meet the maxilla; an Ls point that is an initial point on the side of the upper lip facing to nose of the person to be diagnosed; an Li point on the initial point of the lower lip facing to jaw; a Pog point near the peak point of the jaw and the Sn point at a base of the cavity between the Pog point and the Li point; an N point at a base of the cavity between the forehead and the nose, an Nd point that is the middle of the nasal bridge; a Cm point between the Sn point and the Pn point; an A point between the Ls point and the Sn point; a B point at a base of the cavity between the Li point and the Pog point; a Gn point at the lower edge of the jaw and a Po point at the ear entry; a lower ear point that is the lower point where the ear of the person to be diagnosed meet the face; an upper ear point that is the upper point where the ear meets the face, and a mandible bone in the diagnosis image;
the processing unit is also configured to determine the mandible area in such a way that the edge limits thereof comprise the mandible bone, the Gn point, the Pog point, the B point, the Li point, the lower ear point and the upper ear point, and the maxillary area having a rectangular-like area of which the edge limits comprise the Li point, the point where two lips meet, the Ls point, the A point, the Sn point, the lower ear point and the upper ear point.

3. The system for diagnosis of skeleton syndromes according to claim 1, wherein the processing unit is configured to enable to view an information related to the identity of the matched person on a user interface if the determined area ratio matches with one of the area ratios that belong to recorded persons in the memory unit.

4. A system for diagnosis of skeleton syndromes, wherein skeleton characteristics of a person to be diagnosed are visible from a profile; comprising a user terminal, wherein the user terminal comprises a camera to capture a diagnosis image seen from side of the face of the person to be diagnosed, a processing unit associated with the camera, a user interface controlled by the processing unit and an acceleration sensor; the processing unit is configured to determine a mandible area in the diagnosis image received from the camera; calculate an area ratio that is the ratio of one of the surface area ratio of the maxillary area and the surface area of the jaw area to the other; determine a true vertical line running in parallel to gravitational force in accordance with the measurements received from the acceleration sensor and enable to view it on the user interface; determine in the diagnosis image received from the camera a plurality of points, at least one on the mandible and at least one on the maxilla; determine point distances of determined points to the true vertical line; determine an H angle between a first line passing through two of the determined points and a second point also passing through two of the determined points and having a common point with the first point;
the processing unit is also configured to produce a signal indicating, by means of a machine learning model trained with side profile images that belong to a plurality of healthy persons and a plurality of persons suffering from skeleton syndromes of which the H angles, the point distances and the area ratios are known, whether the diagnosis image contains skeleton syndromes in accordance with the determined H angle, the point distances and the area ratio in the diagnosis image.

5. The system for diagnosis of skeleton syndromes according to the claim 4, wherein the processing unit is configured to determine a G point remained in between the brows of the person to be diagnosed; a Pn point near the peak point of the nose of the person to be diagnosed; an Sn point where the wall between the nostrils of the person to be diagnosed meet the maxilla; an Ls point that is an initial point on the side of the upper lip facing to nose of the person to be diagnosed; an Li point on the initial point of the lower lip facing to jaw; a Pog point near the peak point of the jaw and the Sn point at base of the cavity between the Pog point and the Li point; an N point at base of the cavity between the forehead and the nose, an Nd point that is the middle of the nasal bridge; a Cm point between the Sn point and the Pn point; an A point between the Ls point and the Sn point; a B point at base of the cavity between the Li point and the Pog point; a Gn point at the lower edge of the jaw and a Po point at the ear entry; a lower ear point that is the lower point where the ear of the person to be diagnosed meet the face; an upper ear point that is the upper point where the ear meet the face, and a mandible bone in the diagnosis image;
the processing unit is also configured to determine the mandible area in such a way that the edge limits thereof comprise the mandible bone, the Gn point, the Pog point, the B point, the Li point, the lower ear point and the upper ear point, and the maxillary area having a rectangular-like area of which the edge limits comprise the Li point, the point where two lips meet, the Ls point, the A point, the Sn point, the lower ear point and the upper ear point.

6. The system for diagnosis of skeleton syndromes according to claim 5, wherein the first line is a line passing through the Sn point and the Pog point, and the second line is a line passing through the N point and the Pog point.

7. The system for diagnosis of skeleton syndromes according to claim 4, wherein the processing unit is configured to enable viewing the match result on the user interface if the point distance, the H angle and the area ratio of the diagnosis image match with a person in the training data.

8. A system for diagnosis of skeleton syndromes, wherein skeleton characteristics of a person to be diagnosed are visible from a profile; comprising a user terminal, wherein the user terminal comprises a camera to capture a diagnosis image seen from the side of the face of the person to be diagnosed and a processing unit associated with the camera; the processing unit is configured to transform the received image to a silhouette image, divide the silhouette image into a predetermined number of sub-images and transform the obtained sub-images to a binary vector or matrix, produce a signal related to the skeleton syndromes of the person to be diagnosed by using a deep machine learning model trained with the side profile images in the form of transformed images of persons suffered from skeleton syndromes and healthy persons.
